(19) United States
(12) Patent Application Publication
(10) Pub. No.: US 2025/0050115 A1
(43) Pub. Date: Feb. 13, 2025

(54) ICE-OPTIMIZED LEFT ATRIUM AND LEFT ATRIAL APPENDAGE PACING

(71) Applicant: Boston Scientific Scimed, Inc., Maple Grove, MN (US)

(72) Inventors: Jerry Wang, Edina, MN (US); Joshua Mark Inouye, Brooklyn Park, MN (US); James M. Anderson, Corcoran, MN (US)

(73) Assignee: Boston Scientific Scimed, Inc., Maple Grove, MN (US)

(21) Appl. No.: 18/791,881

(22) Filed: Aug. 1, 2024

Related U.S. Application Data
(60) Provisional application No. 63/531,884, filed on Aug. 10, 2023.

(51) Int. Cl. A61N 1/365; A61N 1/05; A61N 1/368; A61N 1/37; A61N 1/372; A61N 1/375; A61N 1/378

(52) U.S. Cl. CPC A61N 1/36514; A61N 1/0573; A61N 1/36842; A61N 1/3702; A61N 1/37288; A61N 1/3756; A61N 1/378

(57) ABSTRACT

A pacing system configured to sense cardiac activity and to deliver pacing therapy to a patient's heart. The pacing system may comprise a first electrode configured to be positioned in a first chamber of the heart and configured to deliver a first pacing therapy to the first chamber of the heart and a second electrode configured to be positioned in the left atrial appendage and configured to deliver a second pacing therapy to the left atrial appendage. The processing module of the pacing system may be configured to time a delivery of the first pacing therapy based on a first timing fiducial and a delivery of the second pacing therapy based on a determined pacing delay between the first pacing therapy and the second pacing therapy. The determined pacing delay may be configured to maximize a flow of blood into and/or out of the left atrial appendage.

### CROSS-REFERENCE TO RELATED APPLICATIONS

This application claims the benefit of U.S. Provisional Patent Application Ser. No. 63/531,884, filed Aug. 10, 2023, which is incorporated herein by reference.

### TECHNICAL FIELD

The present disclosure generally relates to implantable medical devices and more particularly to implantable cardiac pacemakers.

### BACKGROUND

The left atrial appendage (LAA) is a small organ attached to the left atrium of the heart as a pouch-like extension. In patients suffering from atrial fibrillation, the left atrial appendage may not properly contract with the left atrium, causing stagnant blood to pool within its interior, which can lead to the undesirable formation of thrombi within the left atrial appendage. Thrombi forming in the left atrial appendage may break loose from this area and enter the blood stream. Thrombi that migrate through the blood vessels may eventually plug a smaller vessel downstream and thereby contribute to stroke or heart attack. Clinical studies have shown that the majority of blood clots in patients with atrial fibrillation are found in the left atrial appendage. A continuing need exists for improved medical devices and methods to control thrombus formation within the left atrial appendage of patients suffering from atrial fibrillation.

### SUMMARY

This disclosure provides design, material, manufacturing method, and use alternatives for medical devices.

In a first example, a pacing system configured to sense cardiac activity and to deliver pacing therapy to a patient's heart may comprise a first electrode configured to be positioned in a first chamber of the heart and configured to deliver a first pacing therapy to the first chamber of the heart and a second electrode configured to be positioned adjacent to the left atrial appendage of the heart and configured to deliver a second pacing therapy to the left atrial appendage of the heart. A processing module of the pacing system may be configured to time a delivery of at least part of the first pacing therapy of the first electrode based, at least in part on a first timing fiducial and the processing module of the pacing system may be configured to time a delivery of at least part of the second pacing therapy of the second electrode based, at least in part on a determined pacing delay between the first pacing therapy and the second pacing therapy. The determined pacing delay may be configured to maximize a flow of blood into and/or out of the left atrial appendage.

Alternatively or additionally to any of the examples above, in another example, the first electrode may be coupled to a first lead extending from a pacemaker and the second electrode may be coupled to a second lead extending from the pacemaker.

Alternatively or additionally to any of the examples above, in another example, the second lead may comprise a punch needle, an adhesion growth pad, the second electrode, and an electrical conductor extending proximally from the second electrode.

Alternatively or additionally to any of the examples above, in another example, the punch needle and the adhesion growth pad may be configured to be punched through and compressed against an outer surface of the left atrial appendage.

Alternatively or additionally to any of the examples above, in another example, the first electrode may be coupled to a first lead extending from a pacemaker and the second electrode may be coupled to a leadless cardiac pacemaker.

Alternatively or additionally to any of the examples above, in another example, the pacemaker may be in communication with the leadless cardiac pacemaker.

Alternatively or additionally to any of the examples above, in another example, the first timing fiducial may comprise a heart sound.

Alternatively or additionally to any of the examples above, in another example, the first timing fiducial may comprise a valve opening.

Alternatively or additionally to any of the examples above, in another example, the first timing fiducial may comprise a pressure drop.

Alternatively or additionally to any of the examples above, in another example, the determined pacing delay may be configured to be customized during implantation of the pacing system.

Alternatively or additionally to any of the examples above, in another example, the first chamber of the heart may be a left atrium.

Alternatively or additionally to any of the examples above, in another example, the second electrode may be configured to be positioned in the left atrial appendage.

Alternatively or additionally to any of the examples above, in another example, the second electrode may be configured to be positioned exterior to the left atrial appendage.

Alternatively or additionally to any of the examples above, in another example, the determined pacing delay may be based off of the first timing fiducial.

Alternatively or additionally to any of the examples above, in another example, the determined pacing delay may be based off of the first pacing therapy.

In another example, a method of implanting a pacing system may comprise delivering a first electrode to a first chamber of a heart, delivering a second electrode to a left atrial appendage of the heart, positioning an echocardiography system to measure blood flow into and/or out of the left atrial appendage of the heart, delivering a first pacing therapy to the first chamber of the heart, delivering a second pacing therapy to the left atrial appendage of the heart, the second pacing therapy delivered a period of time after the first pacing therapy, while delivering the first pacing therapy and the second pacing therapy measuring a flow of blood into and/or out of the left atrial appendage, repeatedly delivering the first pacing therapy to the first chamber of the heart, the second pacing therapy to the left atrial appendage of the heart and measuring the flow of blood into and/or out of the left atrial appendage while varying the period of time between the first pacing therapy and the second pacing therapy with each iteration of pacing therapy, selecting an operational period of time between the first pacing therapy and the second pacing therapy which maximizes a flow of blood into and/or out of the left atrial appendage, and providing pacing therapy to the heart using the operational period of time between the first pacing therapy and the second pacing therapy.

Alternatively or additionally to any of the examples above, in another example, repeatedly delivering the first pacing therapy to the first chamber of the heart, the second pacing therapy to the left atrial appendage of the heart and measuring the flow of blood into and/or out of the left atrial appendage while varying the period of time between the first pacing therapy and the second pacing therapy with each iteration of pacing therapy may be performed at implantation of the first and second electrodes.

Alternatively or additionally to any of the examples above, in another example, the operational period of time between the first pacing therapy and the second pacing therapy may be stored in a processing module in communication with the first electrode and the second electrode.

Alternatively or additionally to any of the examples above, in another example, varying the period of time between the first pacing therapy and the second pacing therapy may comprise incrementally increasing the period of time.

Alternatively or additionally to any of the examples above, in another example, a timing of the first pacing therapy may be based, at least in part, off a first timing fiducial.

Alternatively or additionally to any of the examples above, in another example, the operational period of time between the first pacing therapy and the second pacing therapy may be based, at least in part, off a first timing fiducial.

Alternatively or additionally to any of the examples above, in another example, repeatedly delivering the first pacing therapy to the first chamber of the heart, the second pacing therapy to the left atrial appendage of the heart and measuring the flow of blood into and/or out of the left atrial appendage while varying the period of time between the first pacing therapy and the second pacing therapy with each iteration of pacing therapy may be performed during a configuration mode.

Alternatively or additionally to any of the examples above, in another example, providing pacing therapy to the heart using the operational period of time between the first pacing therapy and the second pacing therapy may occur in response to a predetermined detected condition.

Alternatively or additionally to any of the examples above, in another example, the predetermined detected condition may be a detected atrial fibrillation and/or a detected pressure drop.

In another example, a method of implanting a pacing system may comprise delivering a first electrode to a first chamber of a heart, delivering a second electrode to a left atrial appendage of the heart, advancing an echocardiography system adjacent to the left atrial appendage of the heart, ablating a region of heart tissue between the first electrode and the second electrode, delivering a first pacing therapy to the first chamber of the heart, delivering a second pacing therapy to the left atrial appendage of the heart, the second pacing therapy delivered a period of time after the first pacing therapy, while delivering the first pacing therapy and the second pacing therapy measuring a flow of blood into and/or out of the left atrial appendage, repeatedly delivering the first pacing therapy to the first chamber of the heart, the second pacing therapy to the left atrial appendage of the heart and measuring the flow of blood into and/or out of the left atrial appendage while varying the period of time between the first pacing therapy and the second pacing therapy with each iteration of pacing therapy, selecting an operational period of time between the first pacing therapy and the second pacing therapy which maximizes a flow of blood into and/or out of the left atrial appendage, and providing pacing therapy to the heart using the operational period of time between the first pacing therapy and the second pacing therapy.

Alternatively or additionally to any of the examples above, in another example, providing pacing therapy to the heart using the operational period of time between the first pacing therapy and the second pacing therapy may occur with each heartbeat.

In another example, a method of implanting a pacing system in a heart may comprise a) detecting a first pacing fiducial, b) delivering a first electrode to a left atrial appendage of the heart following a delay after the pacing fiducial, c) advancing an echocardiography system adjacent to the left atrial appendage of the heart, d) delivering a first pacing therapy to the left atrial appendage of the heart, e) measuring a flow of blood into and/or out of the left atrial appendage following the first pacing therapy, f) repeatedly delivering the first pacing therapy to the first chamber of the heart while varying the delay to identify a default delay associated with a maximum flow, g) configuring a pacing system including the first electrode using the default delay from the first pacing fiducial. Alternatively or additionally, in another example, the method may further comprise selecting a second pacing fiducial, and repeating steps a), b), d), c), f) and g) using the second pacing fiducial in place of the first pacing fiducial.

Alternatively or additionally to any of the examples above, in another example, the method may further comprise ablating a region of heart tissue adjacent the left atrial appendage.

In any of the preceding examples, “flow” or “blood flow” may be measured or parameterized as, for example and without limitation, a flow velocity, a peak velocity, an average velocity, flow volume, ejection fraction, or other suitably related parameter. Further, in any of the preceding examples, echocardiography may include, but is not limited to, intracardiac echocardiography (ICE), transesophageal echocardiography (TEE), transthoracic echocardiography (TTE), and may be performed at the time of or after implantation of the pacing system and used to optimize the pacing delay or other aspect of the device or method.

The above summary of some embodiments is not intended to describe each disclosed embodiment or every implementation of the present disclosure. The Figures, and Detailed Description, which follow, more particularly exemplify these embodiments

### BRIEF DESCRIPTION OF THE DRAWINGS

The disclosure may be more completely understood in consideration of the following detailed description of various embodiments in connection with the accompanying drawings, in which:

FIG. 1 is a partial cross-sectional view of certain elements of a heart and some immediately adjacent blood vessels;

FIG. 2 is a schematic view of an illustrative leadless cardiac pacemaker (LCP);

FIG. 3 is a schematic view of another medical device (MD);

FIG. 4 is a graphical representation of an illustrative electrocardiogram (ECG) showing a temporal relationship between electrical signals of the heart and mechanical indications of contraction of the heart;

FIG. 5 is a plan view of an illustrative medical device system;

FIG. 6 is a plan view of another illustrative medical system;

FIG. 7 is a plan view of another illustrative medical system;

FIG. 8 is a schematic cross-sectional view of a heart with an implanted medical device and an illustrative intracardiac echocardiography system;

FIG. 9A is a schematic graph of an illustrative device stimulation protocol;

FIG. 9B is a schematic graph of the flow velocity versus the pacing delay of a pacing system; and

FIGS. 10A-10C depict schematic views of an alternative illustrative lead being delivered to the left atrial appendage.

While aspects of the disclosure are amenable to various modifications and alternative forms, specifics thereof have been shown by way of example in the drawings and will be described in detail. It should be understood, however, that the intention is not to limit aspects of the disclosure to the particular embodiments described. On the contrary, the intention is to cover all modifications, equivalents, and alternatives falling within the spirit and scope of the disclosure.

### DETAILED DESCRIPTION

The following description should be read with reference to the drawings, which are not necessarily to scale, wherein like reference numerals indicate like elements throughout the several views. The detailed description and drawings are intended to illustrate but not limit the claimed disclosure. Those skilled in the art will recognize that the various elements described and/or shown may be arranged in various combinations and configurations without departing from the scope of the disclosure. The detailed description and drawings illustrate example embodiments of the claimed disclosure. However, in the interest of clarity and ease of understanding, while every feature and/or element may not be shown in each drawing, the feature(s) and/or element(s) may be understood to be present regardless, unless otherwise specified.

For the following defined terms, these definitions shall be applied, unless a different definition is given in the claims or elsewhere in this specification.

All numeric values are herein assumed to be modified by the term “about,” whether or not explicitly indicated. The term “about”, in the context of numeric values, generally refers to a range of numbers that one of skill in the art would consider equivalent to the recited value (e.g., having the same function or result). In many instances, the term “about” may include numbers that are rounded to the nearest significant figure. Other uses of the term “about” (e.g., in a context other than numeric values) may be assumed to have their ordinary and customary definition(s), as understood from and consistent with the context of the specification, unless otherwise specified.

The recitation of numerical ranges by endpoints includes all numbers within that range, including the endpoints (e.g., 1 to 5 includes 1, 1.5, 2, 2.75, 3, 3.80, 4, and 5).

Although some suitable dimensions, ranges, and/or values pertaining to various components, features and/or specifications are disclosed, one of skill in the art, incited by the present disclosure, would understand desired dimensions, ranges, and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that in order to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For simplicity and clarity purposes, not all elements of the disclosure are necessarily shown in each figure or discussed in detail below. However, it will be understood that the following discussion may apply equally to any and/or all of the components for which there are more than one, unless explicitly stated to the contrary. Additionally, not all instances of some elements or features may be shown in each figure for clarity.

Relative terms such as “proximal”, “distal”, “advance”, “retract”, variants thereof, and the like, may be generally considered with respect to the positioning, direction, and/or operation of various elements relative to a user/operator/manipulator of the device, wherein “proximal” and “retract” indicate or refer to closer to or toward the user and “distal” and “advance” indicate or refer to farther from or away from the user. In some instances, the terms “proximal” and “distal” may be arbitrarily assigned in an effort to facilitate understanding of the disclosure, and such instances will be readily apparent to the skilled artisan. Other relative terms, such as “upstream”, “downstream”, “inflow”, and “outflow” refer to a direction of fluid flow within a lumen, such as a body lumen, a blood vessel, or within a device.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to effect the particular feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is exemplary only. In some embodiments, alterations of and deviations from previously-used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

A normal, healthy heart induces contraction by conducting intrinsically generated electrical signals throughout the myocardium. These intrinsic signals cause the muscle cells or tissue of the heart to contract in a coordinated manner. These contractions force blood out of and into the heart, providing circulation of the blood throughout the rest of the body. Many patients suffer from cardiac conditions that affect the efficient operation of their hearts. For example, some hearts develop diseased tissue that no longer generate or efficiently conduct intrinsic electrical signals. In some examples, diseased cardiac tissue may conduct electrical signals at differing rates, thereby causing an unsynchronized and inefficient contraction of the heart. In other examples, a heart may generate intrinsic signals at such a low rate that the heart rate becomes dangerously low. In still other examples, a heart may generate electrical signals at an unusually high rate, even resulting in cardiac tachycardia or fibrillation. In some cases, such an abnormality can develop into a fibrillation state, where the contraction of the patient's heart chambers is almost completely de-synchronized and the heart pumps very little to no blood. Implantable medical devices (e.g., pacing devices) which may be configured to determine occurrences of such cardiac abnormalities or arrhythmias and deliver one or more types of electrical stimulation therapy to patient's hearts, may help to terminate or alleviate these and other cardiac conditions.

The occurrence of thrombi in the left atrial appendage (LAA) during atrial fibrillation may be due to stagnancy of blood pooling in the LAA. The pooled blood may still be pulled out of the left atrium by the left ventricle, however less effectively due to the irregular contraction of the left atrium caused by atrial fibrillation. Therefore, instead of an active support of the blood flow by a contracting left atrium and left atrial appendage, filling of the left ventricle may depend primarily or solely on the suction effect created by the left ventricle. However, the contraction of the left atrial appendage may not be in sync with the cycle of the left ventricle. For example, contraction of the left atrial appendage may be out of phase up to 180 degrees with the left ventricle, which may create significant resistance to the desired flow of blood. Further still, most left atrial appendage geometries are complex and highly variable, with large irregular surface areas and a narrow ostium or opening compared to the depth of the left atrial appendage. These aspects as well as others, taken individually or in various combinations, may lead to high flow resistance of blood out of the left atrial appendage.

In an effort to reduce the occurrence of thrombi formation within the left atrial appendage and prevent thrombi from entering the blood stream from within the left atrial appendage, it may be desirable to develop medical devices and/or systems that increase contractility in the LAA and/or increase blood flow into and/or out of the LAA, thereby leading to less stasis and thrombosis within the LAA. Example medical devices and/or systems that increase contractility in the LAA and/or increase blood flow into and/or out of the LAA are disclosed herein.

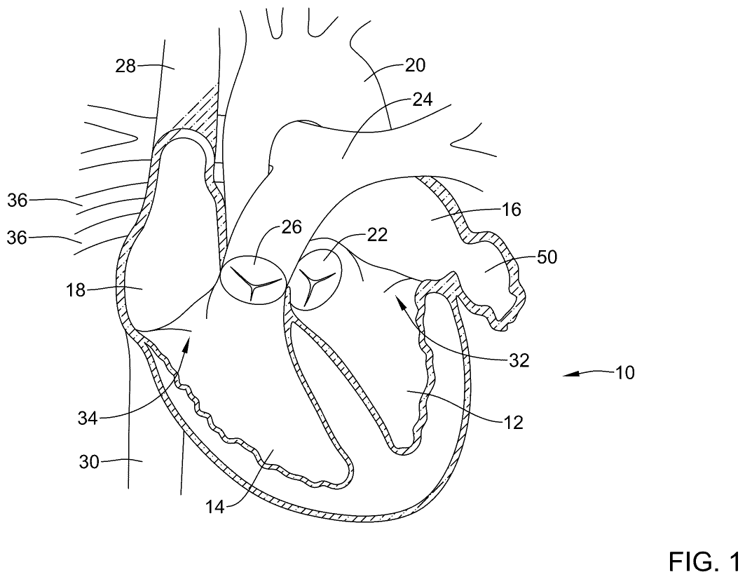

FIG. 1 is a partial cross-sectional view of certain elements of a heart 10 and some immediately adjacent blood vessels. The heart 10 may include the left ventricle 12, the right ventricle 14, the left atrium 16, and the right atrium 18. The aortic valve 22 is disposed between the left ventricle 12 and the aorta 20. The pulmonary or semi-lunar valve 26 is disposed between the right ventricle 14 and the pulmonary artery 24. The superior vena cava 28 and the inferior vena cava 30 return blood from the body to the right atrium 18. The tricuspid valve 34 is disposed between the right atrium 18 and the right ventricle 14. Pulmonary veins 36 return blood from the lungs to the left atrium 16. The mitral valve 32 is disposed between the left atrium 16 and the left ventricle 12. The left atrial appendage (LAA) 50 is attached to and in fluid communication with the left atrium 16.

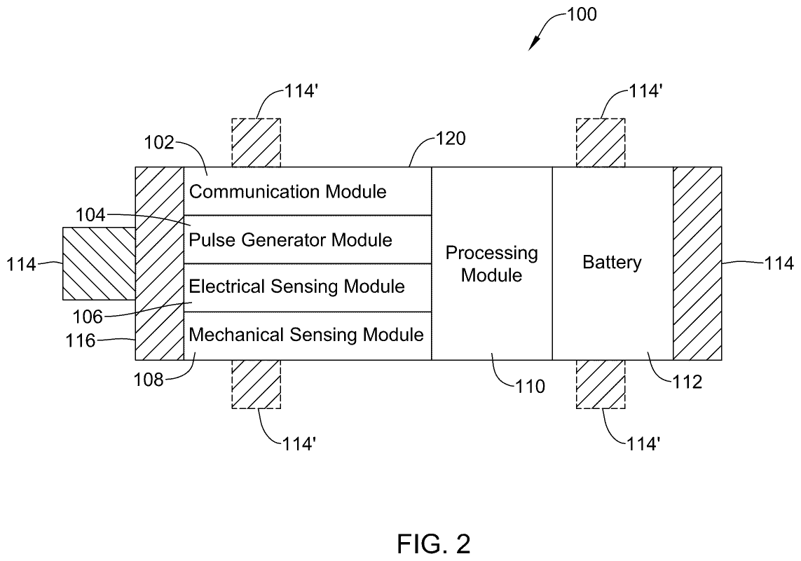

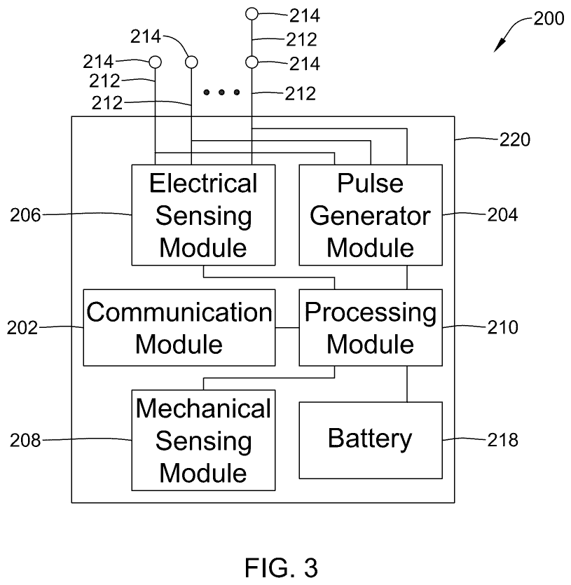

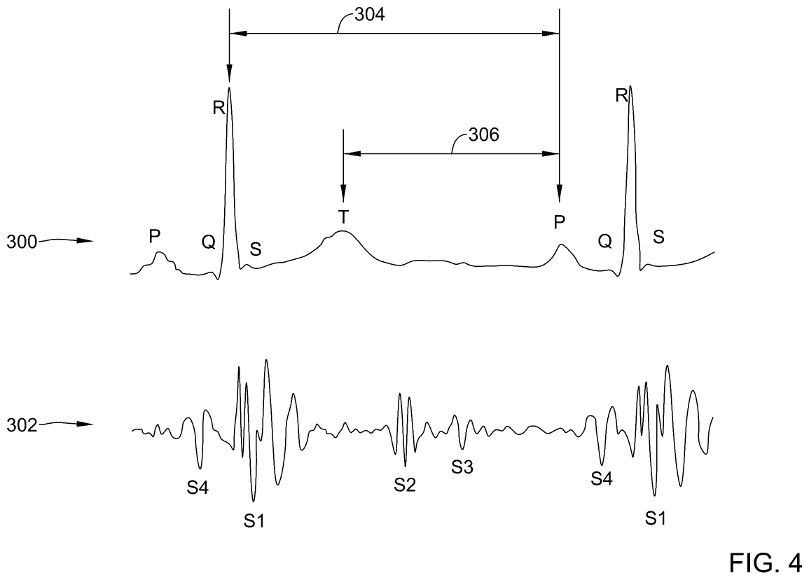

FIGS. 2-3 show illustrative implantable medical devices, and FIG. 4 shows illustrative ECG 300 and heart sounds 302 signals, illustrating details of each and a temporal relationship therebetween. A more detailed discussion of FIGS. 2-4 follows, below. For introductory purposes, it may be understood that a leadless cardiac pacemaker (LCP) is shown in FIG. 2, including an anchoring section 116 that may be use to anchor the LCP in a position in or on the heart, such as by attachment to the heart wall. The LCP 100 includes electrodes 114, 114′ that can be used to sense cardiac signals via an electrical sensing module 106, processed with a processing module 110. In response to the sensed signals (or lack thereof), a pulse generator module 104 uses power from battery 112 to issue electrical pulses via electrodes 114, 114′ to pace the heart, causing contraction of the myocardium. In some illustrative examples, the LCP 100 may be implanted in, on, or adjacent to the LAA, and used to induce blood flow from the LAA to prevent excessive pooling of blood in the LAA which can cause thrombi to form. A pacemaker is shown in FIG. 3, again including an electrical sensing module 206, processing module 210, and pulse generator module 204, which can be used to sense the heart rhythm and issue pacing pulses via electrodes on one or more leads 214. In some illustrative examples, the leads are positioned in, on, or adjacent to the LAA, and used to induce blood flow from the LAA to prevent excessive pooling of blood in the LAA which can cause thrombi to form.

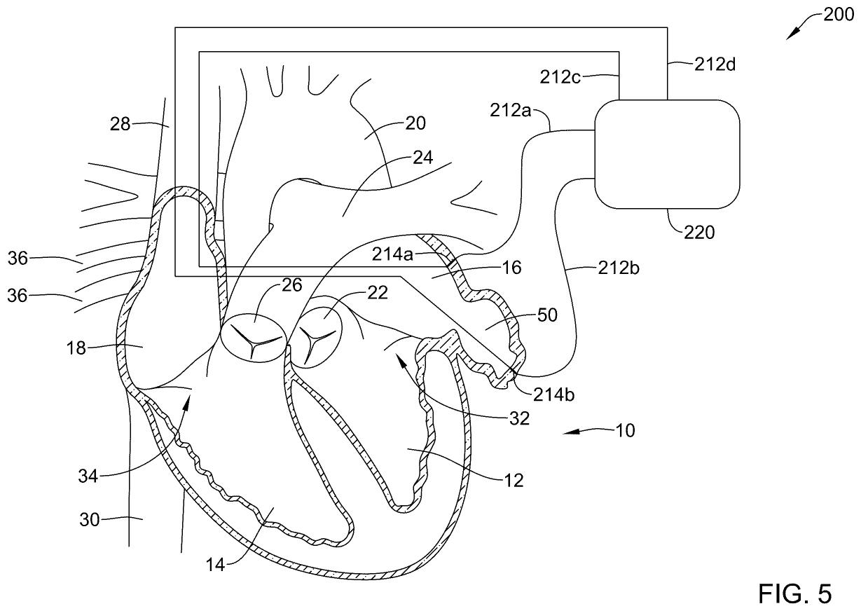

FIGS. 5-7 show illustrative medical device systems that may be configured to operate according to techniques disclosed herein. Other example medical device systems may include additional or different medical devices and/or configurations. For instance, other medical device systems that are suitable to operate according to techniques disclosed herein may include additional LCPs implanted within the heart. Another example medical device system may include a plurality of LCPs without other devices such as the MD 200. In yet other examples, the configuration or placement of the medical devices, leads, and/or electrodes may be different from those depicted in FIGS. 5-7. Accordingly, it should be recognized that numerous other medical device systems, different from those depicted in FIGS. 5-7, may be operated in accordance with techniques disclosed herein. As such, the examples shown in FIGS. 5-7 should not be viewed as limiting in any way.

FIG. 5 is a plan view of the example MD 200 configured to be implanted within the chest (or other location) with leads 212a, 212b implanted within or on the left atrium 16 and the LAA 50. It is contemplated that the second lead 212b may be positioned at a distal site of the LAA 50. However, this is not required. Other implant locations may be used as desired. It is further contemplated that more than one lead may be affixed to the left atrium 16 and/or more than one lead may be affixed to the LAA 50. It is further contemplated that additional leads may be affixed to other portions of the heart 10. In FIG. 5, the leads 212a, 212b are shown extending onto tissue of the heart 10 to position one or more electrodes 214a, 214b in the myocardium. An epicardial approach is shown for leads 212a, 212b. Rather than an epicardial implantation, another approach may be as shown with leads 212c and 212d, in which the leads are threaded through the venous system, to the right atrium 18, then pass through a trans-septal puncture to the left atrium 16, and are anchored in the atrial wall adjacent to or in the LAA 50. The system may be configured to stimulate both the left atrium 16 and the LAA 50 to increase the flow of blood into and/or out of the LAA 50.

In the system of FIG. 5, pacing pulses may be delivered in various ways. Pacing pulses may be issued between electrodes 214a and 214b, if desired, creating an electrical field across the relatively larger area therebetween. Alternatively, at least electrode 214b may be a compound electrode such as having a tip electrode and ring electrode located a few millimeters proximal of the tip electrode, and pacing pulses can be issued between tip and ring. With the compound electrode at 214b, electrode 214a may be used to sense whether biological signals are passing along the myocardium (retrograde) to the location of electrode 214a, and/or to determine the size of any pacing artifact at the location of electrode 214a, when the compound electrode is used to issue pacing pulses. Both electrodes 214a and 214b may be compound electrodes, and pacing stimulus may be applied at both sites, for example in a synchronized fashion (simultaneously or with a set and/or adjustable delay therebetween, for example). Other ways of using the electrodes are discussed below.

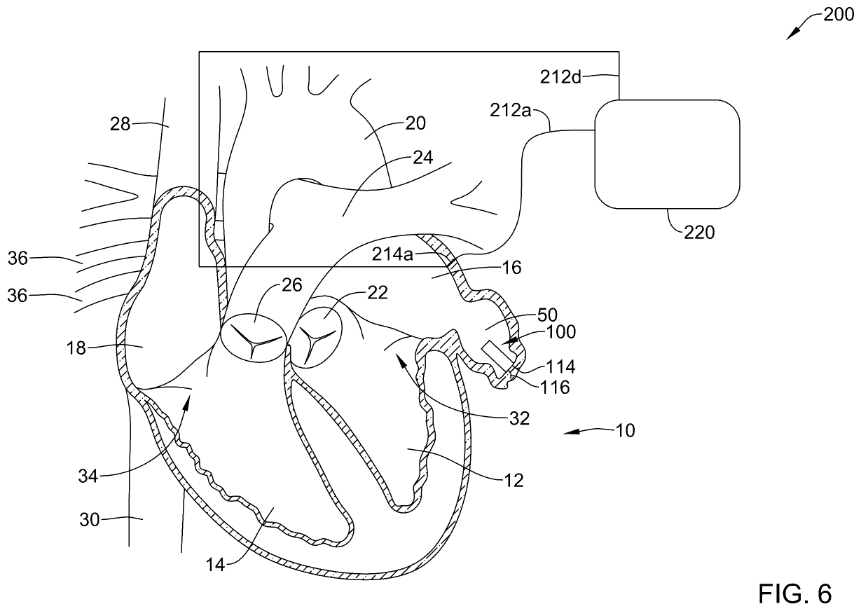

FIG. 6 is a plan view of an example with an LCP 100 be implanted in the LAA 50. In addition, in the example, a second MD 200 may be configured to be implanted within the chest (or other location) with a transvenous lead 212d implanted within the left atrium 16; alternatively, epicardial lead 212a may by implanted, or no left atrial lead may be used, as desired. It is contemplated that the LCP 100 may be positioned at a distal site of the LAA 50. However, this is not required. Other implant locations may be used as desired, in, on, or adjacent to the LAA 50. It is further contemplated that more than one lead may be affixed to the left atrium 16 and/or more than one lead and/or more than one LCP 100 may be affixed to the LAA 50. It is further contemplated that additional leads may be affixed to other portions of the heart 10. While not explicitly shown, in some cases, the LCP 100 may be implanted in contact with an exterior surface of the LAA 50. In FIG. 6, the lead 212a and the fixation mechanism 116 of the LCP 100 are shown extending into tissue of the heart 10 to position one or more electrodes 214a, 114 into the endocardial tissue. The system may be configured to stimulate both the left atrium 16 and the LAA 50 to increase the flow of blood into and/or out of the LAA 50.

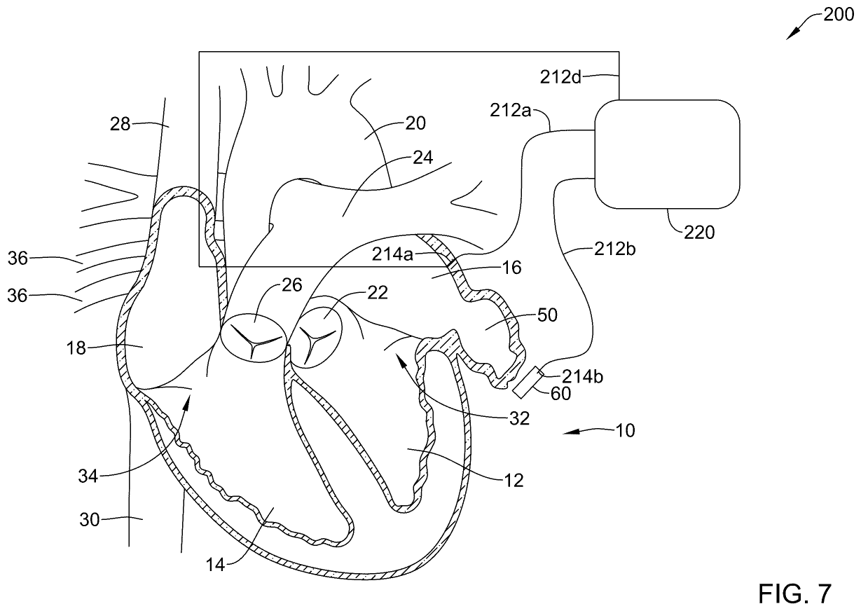

FIG. 7 is a plan view of the example MD device 200 configured to be implanted within the chest (or other location) with leads 212a or 212d implanted within the left atrium 16 and lead 212b extending to nearby cardiac or skeletal muscle 60 external to the LAA 50. In FIG. 7, the leads 212a or 212d are shown extending into tissue of the heart 10 to position one or more electrodes 214a into the myocardium. While not explicitly shown, an additional lead from the MD 200 or an LCP 100 may be positioned in contact with the LAA 50. The system may be configured to stimulate both the left atrium 16 and tissue adjacent to the LAA 50 to increase the flow of blood into and/or out of the LAA 50.

It is further contemplated that the LCP 100 may be guided toward the heart 10 via the inferior vena cava 30 to the right atrium 18. The LCP may then be delivered transeptally to the LAA 50. In other examples, the LCP may be delivered from a femoral approach, without needing trans-septal access. Visualization, such as by contrast injection, may be used to aid the placement of the LCP, as needed. In some examples, the pacing systems of FIGS. 5-7 may omit the lead 212a extending to the left atrium 16. For example, the LAA 50 may be stimulated independently or in response to another pacing system.

The pacing systems of FIGS. 5-7 may be configured to provide pacing pulses to the left atrium 16 and the LAA 50 (or a region adjacent thereto) concomitantly. In some cases, pacing therapy may be delivered to the left atrium 16 and the LAA 50 sequentially. For example, in some cases, the left atrium 16 may be stimulated by a first pacing therapy and after a predetermined delay time period, the LAA 50 may be stimulated by a second pacing therapy. It is contemplated that the delay between stimulations/pacing therapies may be selected to optimize or maximize the flow of blood into and/or out of the LAA 50. The flow of blood into and/or out of the LAA 50 may be measured as a flow velocity, a peak velocity, an average velocity, flow volume, ejection fraction, etc. Echocardiography, including, but not limited to, intracardiac echocardiography (ICE), transesophageal echocardiography (TEE), transthoracic echocardiography (TTE), may be performed at the time of or after implantation of the pacing system and used to optimize the pacing delay between the left atrium 16 and the LAA 50. For example, the pacing delay may be customized for each patient.

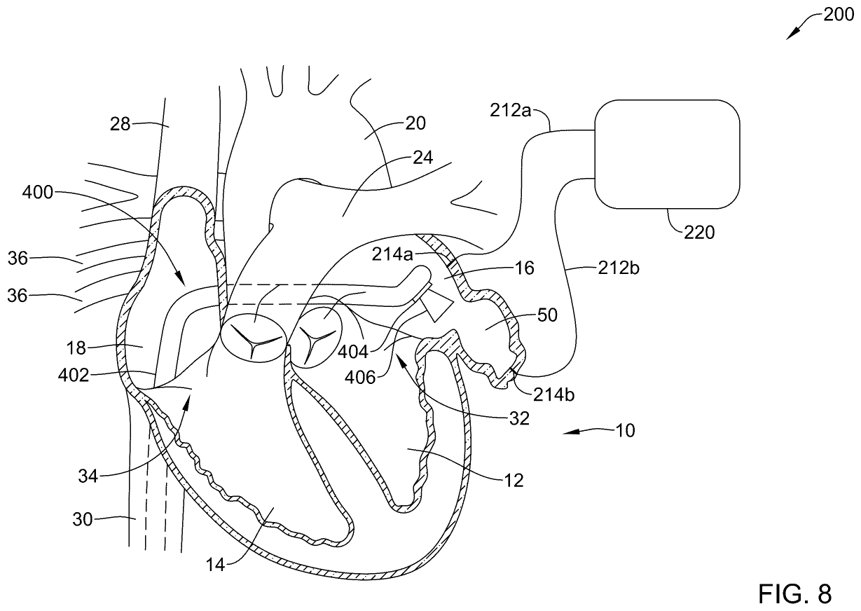

FIG. 8 is a schematic cross-sectional view of a heart 10 with an implanted MD 200 and an illustrative ICE system 400. While FIG. 8 is described with respect to an ICE system, other echocardiography systems such as, but not limited to TEE or TTE may be used, as desired. The ICE system 400 may include an elongate shaft 402, such as, but not limited to, a catheter shaft and an ultrasound device 404 disposed adjacent to a distal end of the elongate shaft 402. It is contemplated that while not explicitly shown the ICE system 400 may include other features, such as steering wires, guide sheaths, etc. The ICE system 400 may be a radial or rotational system or a phased array system, as desired.

The implanted MD 200 may include leads 212a, 212b implanted within the left atrium 16 and the LAA 50. Transvenous leads are omitted from the Figure to allow the ICE system 400 to be more readily seen, but it should be understood that transvenous leads 212c, 212d, shown above, may be used in place of the epicardial leads shown. In FIG. 8, the leads 212a, 212b are shown extending into tissue of the heart 10 to position one or more electrodes 214a, 214b into the endocardial tissue. It should be understood that the imaging and pacing time delay selection techniques described with respect to FIG. 8 may be applied to any of the pacing systems described herein. For example, the pacing system may include a MD 200 and an LCP 100. After implantation of the MD 200, the ICE system 400 may be guided toward the heart 10 via the inferior vena cava 30 to the right atrium 18. As viewed from an anterior side of a patient, a distal portion of the ICE system 400 may include a right-hand curve near its distal end, such that the ICE system 400 curves toward a wall (e.g., the septum) between the right atrium 18 and the left atrium 16, although this is not required. The wall (septum) between the right atrium 18 and the left atrium 16 is punctured and the ICE system 400 is delivered through the wall to a position adjacent the LAA 50. This is just one example. It is contemplated that the ICE system 400 may be delivered to the left atrium 16 using other paths, as desired.

Once the ICE system 400 is in place, the ultrasound device 404 may be positioned relative to and/or directed towards the LAA and activated. The ultrasound device 404 may emit high-frequency sound waves 406 towards the LAA 50. The sound waves 406 may be used to measure the flow of blood as a velocity, volume, and/or ejection fraction into and out of the LAA 50 via, for example, color doppler. Once the ICE system 400 is in place, the MD 200 may be placed into a configuration mode. For example, the clinician may use a display or user input to select a configuration mode for the MD 200. When more than one implantable device is provided, as in the system of FIG. 6, each of the implantable devices 100, 200 may be placed into a configuration mode. Once in the configuration mode, the processing module 210 of the MD 200 may be configured to vary a time delay between the delivery of a pacing pulse at each lead (any of 212a, 212b, 212c, 212d, and/or the electrodes of the LCP 100) whether under the direction of an external programmer or independently in an automated mode. In some cases, the processing module 210 may automatically vary the time delay between the delivery of a pacing pulse at each lead. In other examples, the processing module 210 may be configured to vary the time delay between activation of each lead in accordance with a user input received at the display or user input.

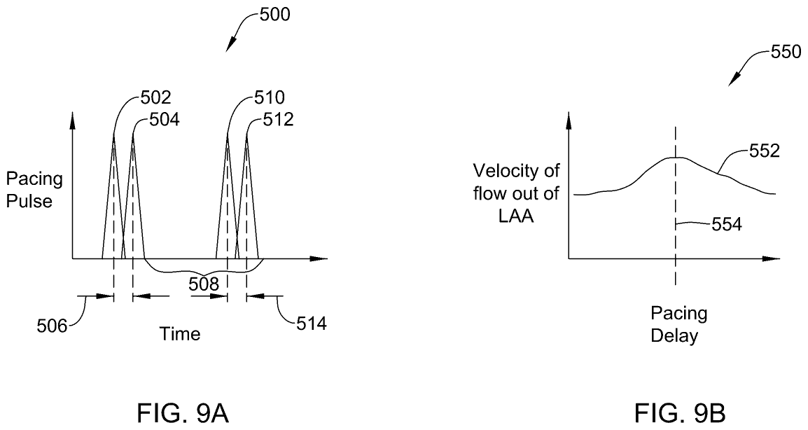

Referring additionally to FIG. 9A, which illustrates a schematic graph 500 of an illustrative pacing device stimulation protocol, a first lead may receive a first pacing therapy 502 at a first time point and a second lead may have a first pacing therapy 504 at a second time point. The second time point may be a first length of time 506 after the first time point. It is contemplated that each lead may be activated once over the course of a single heartbeat 508. However, it is not required that the leads be activated with every heartbeat. It is further contemplated that the first lead may be activated in response to a predetermined timing fiducial. The time delay between activation of the first lead and activation of the second lead may be incrementally increased over a series of successive heartbeats. Said differently, the pacing therapy to the first lead and the pacing therapy to the second lead may be repeatedly delivered while varying the period of time between the pacing therapies. It is contemplated that the incremental increases may be a fixed amount of time or a varying amount of time, as desired, and may occur with each heart beat/pacing output, or changes may be made every “N” heart beats, where N may be 1, 2, 3, 4, 5, etc. or larger number, as desired. The first lead may have a second activation 510 at a third time point and the second lead may have a second activation 512 at a fourth time point. The fourth time point may be second length of time 514 after the third time point, the second length of time 514 greater than the first length of time 506.

As the processing module 210 is adjusting the pacing delay between the first and second leads, the ICE system 400 may be determining and/or recording a flow of blood into and/or out of the LAA 50 for each iteration of the pacing therapy (e.g., as a peak velocity, average velocity, flow volume, ejection fraction, etc.). The processing module 210 or an external computing device may record the flow of the blood into and/or out of the LAA 50 and the corresponding pacing delay. In some cases, the flow of the blood and the pacing delay may be recorded and/or displayed as a graph 550, as shown in FIG. 9B, a table, or other data recording system. The graph 550 may show a curve 552 of the flow velocity of the blood into and/or out of the LAA versus the pacing time delay between the first lead and the second lead. As shown in FIG. 9B, the flow velocity of the blood into and/or out of the LAA 50 may be maximized at a particular pacing time delay, as shown at line 554 of FIG. 9B. While FIG. 9B illustrates the flow of blood as a flow velocity, other variables such as, but not limited to, a peak velocity, average velocity, flow volume, ejection fraction may be used to maximize or optimize the flow of blood from the LAA 50. The processing module 210 may be configured to store an operational period of time between pacing therapies or an operational pacing time delay at which the flow of the blood into and/or out of the LAA 50 is maximized.

In some cases, the processing module 210 (or external computing device) may automatically determine and store the operational period of time between pacing therapies or an operational pacing time delay which the flow of the blood into and/or out of the LAA 50 is maximized. In other cases, the user may input the pacing time delay at which the flow of the blood into and/or out of the LAA 50 is maximized into the external computing device to be relayed to the processing module 210 of the MD 200. The processing module 210 may then exit the configuration mode and enter an operational mode either automatically or in response to a user input and the ICE system 400 may be removed from the body. It is contemplated that when more than one pacing device is provided, the operational period of time between pacing therapies or an operational pacing time delay may be provided to each processing module, or one of the pacing devices may be a dominant device issuing operation commands to one or more sub-devices.

Once in the operational mode, the processing module 210 may be configured to deliver a pacing therapy using the parameters determined during the configuration mode procedure. For example, the first lead may be activated in response to a determined pacing fiducial marker and the second lead may be activated “x” seconds after the first lead. It is contemplated that the predetermined pacing time delay “x” may be in the range of milliseconds, for example, in the range of 0.1 millisecond up to about 50 milliseconds.

In addition to maximizing a blood flow into and/or out of the LAA 50, polarizing and/or depolarizing the LAA 50 at a distal site thereof may create motion and/or instability which may additionally reduce or prevent thrombus formation. In some examples, the MD 200 may be configured to deliver pacing therapy in response to a predetermined condition. For example, the MD 200 may not pace the LAA every heartbeat, which may extend the longevity of the MD 200. In some cases, the MD 200 may be configured to pace when atrial fibrillation is detected. As noted above when atrial fibrillation is detected, the LAA 50 may not contract properly and stagnant blood may pool therein. Delivering pacing energy to the left atrium 16 and the LAA 50 may increase the flow velocity of the blood entering and exiting the LAA 50 as well as increase movement thereof to reduce stagnation and thus clots. In another example, pacing therapy may be delivered in response to the pressure within the left atrium 16, the LAA 50, or other areas of the heart 10. For example, when higher pressures are present, less stimulation of the atrium 16 and/or LAA 50 may be required. For example, the duty cycle of LAA pacing may vary as a function of one or more fiducials including the presence or absence of atrial fibrillation or atrial flutter, presence or absence of a sensed P-wave (absence of which may indicate an undetected atrial fibrillation or flutter), intra-atrial blood pressure above or below a threshold, presence or absence of any other arrhythmia, the presence of irregular heart sounds, the absence of any one of the heart sounds identified below in the discussion of FIG. 4, etc. Multiple such conditions may be monitored. Duty cycle of LAA pacing may vary linearly or in a stepwise fashion, such as shown here:
If regular P-wave, duty cycle of LAA pacing=10%
If P-wave not detected, duty cycle of LAA pacing=33%
If atrial fibrillation detected, duty cycle of LAA pacing=100%
   Other configurations may be used. In some examples, a pressure differential in the left atrium may be detected to determine a pressure change from minimum to maximum left atrial pressure during cardiac cycles. When the pressure differential is relatively higher, then the duty cycle of LAA pacing may be relatively lower (less than 50%, for example), and when the pressure differential is relatively lower, the duty cycle of LAA pacing may be relatively higher (more than 50%, for example).

Pacing therapy applied to the LAA may use a plurality of different LAA pacing fiducials. An LAA pacing fiducial as used herein is an event that triggers an LAA pacing output, either immediately or after a delay. Some examples may use as an LAA pacing fiducial the detection of the opening of the mitral valve 32, such as by monitoring heart sounds, for example, S3. In another example, the LAA pacing fiducial may be detection of the S4 heart sound. Another LAA pacing fiducial may be detection of a drop in atrial pressure, or downward slope in atrial pressure. Still another LAA pacing fiducial may be detection of a pacing pulse by another implanted system, or delivery of a pace therapy by the same implanted system as the one issuing LAA pacing therapy. Yet another LAA pacing fiducial may be an electrical event such as P-wave onset, or P-wave peak. In a multi-chamber pacing system, for example, a lead having electrodes in the right atrium may detect onset of right atrial depolarization (P-wave onset), which may be used as the fiducial for LAA pacing.

Once an LAA pacing fiducial is detected, an LAA pace pulse may be delivered immediately or after a predetermined delay. If both LAA and left atrial pace pulses are separately delivered, the LAA pacing fiducial can be used to trigger first one, then the other pace output. In some examples, the configuration mode procedures described above may be performed using a plurality of different LAA pacing fiducials, with the result being a plurality of different optimized LAA to left atrium pacing delays. For example, an LAA pacing system may be configured to use either a detected mitral valve opening event or P-wave detection in the right atrium as a pacing fiducial, and the delays to first pace pulse, and from first pace pulse to second pace pulse, may be selected according to which LAA pacing fiducial triggers pace outputs.

It is contemplated that a region of the atrium 16 and/or LAA 50 may be ablated prior to providing pacing therapy to the atrium 16 and/or the LAA 50. For example, ablation may isolate the LAA 50 from the rest of the atrial neural path. This may insulate the LAA 50 from atrial fibrillation. However, ablation is not required. When ablation is performed, pacing pulses may be provided with every heartbeat as the LAA 50 is isolated from the atrial neural path and not receiving the intrinsically generated electrical signals of the heart 10. Alternatively, pacing pulses may be provided at a less than 100% duty cycle after the LAA has been electrically isolated from the cardiac neural system, using, for example, any of the above noted factors for raising or lowering duty cycle.

In another example, the illustration of FIGS. 9A-9B may be used in a different method. Here, elements 502 and 510 may be understood instead as pacing fiducials, that is, detected events that are used to trigger an LAA pacing output at 504 and 512. Here, only one pace output is generated in this example, directly in the LAA. The time delay from detection of a pacing fiducial 502, 510 to issuance of an LAA pace output at 504, 513 is then varied to create a graph as shown in FIG. 9B, and the delay that maximizes flow velocity can then be selected as the delay to use from pacing fiducial to LAA pace output. This method may be repeated for a plurality of different pacing fiducials. Still further, the method may also be repeated for a two-pace system by first optimizing the delay from pacing fiducial to initial pace output, and then optimizing the delay between two pace outputs, if desired.

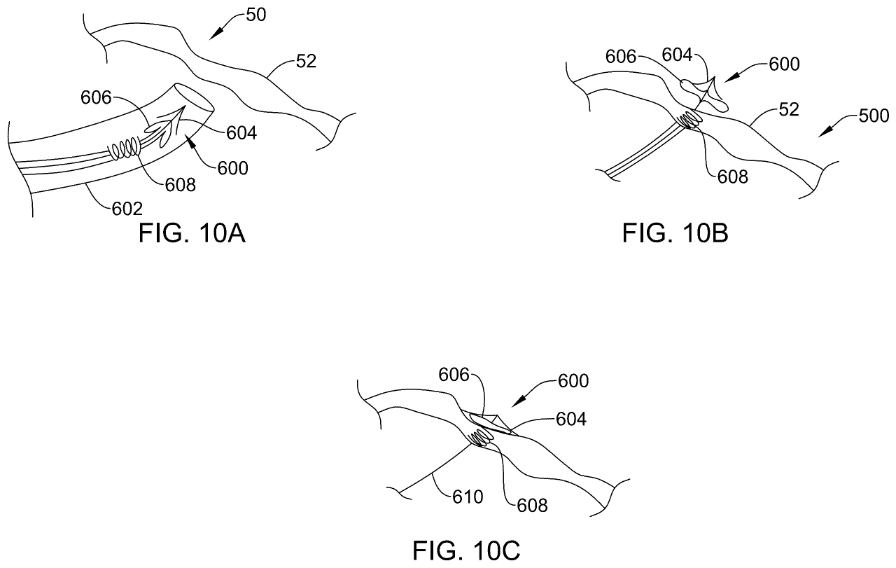

In some examples, a traditional helical lead may not be well suited for use in the LAA 50. It is contemplated in some examples, a lead for use in the LAA 50 may be configured to extend through and seat on an exterior surface of the LAA. FIGS. 10A-10C depict schematic views of an alternative illustrative lead 600 being delivered to the LAA 50. In FIG. 10A, the lead 600 is advanced to the target location within a delivery sheath 602. In the illustrated embodiment, the target location may be a wall 52 of the LAA 50. However, this is not required. The illustrative lead 600 may be used in other locations of the heart 10, as desired. It is contemplated that delivery sheath 602 may be guided toward the heart 10 via the inferior vena cava 30 to the right atrium 18. The delivery sheath 602 may extend through a puncture in the septum between the right atrium 18 and the left atrium 16 to enter the LAA 50.

Once the delivery sheath 602 is adjacent to the target implantation location, a punch needle 604 of the lead 600 may be advanced distally beyond a distal end of the delivery sheath 602 and through the wall 52, as shown in FIG. 10B. An adhesion growth pad 606 may be positioned between the punch needle 604 and an outer surface of the wall 52. The adhesion growth pad 606 may be configured to encourage tissue growth to further secure the lead 600 relative to the tissue. An electrode 608 may be proximally spaced from the punch needle 604 such that when the punch needle 604 extends through the wall 52, the electrode 608 is in contact with the endocardial tissue. A conductive member 610 may extend proximally from the electrode 608 to the pacing device. Finally, the lead 600 may be proximally retracted to radially expand the punch needle 604 and compress the adhesion growth pad 606 against the tissue, as shown in FIG. 10C. The delivery sheath 602 and any other delivery aids may be proximally retracted from the body.

Still another approach may be to enter the thorax through an intercostal space, and advancing the lead into a position adjacent the LAA from the exterior surface thereof. The above description of lead anchoring in FIGS. 10A-10C may be used, but with the placement system/catheter 602 being placed on the outside of the LAA.

Following is a more detailed discussion of FIGS. 2-4. FIG. 2 depicts an illustrative leadless cardiac pacemaker (LCP) that may be implanted into a patient and may operate to prevent, control, or terminate cardiac arrhythmias in patients by, for example, appropriately employing one or more therapies (e.g. anti-tachycardia pacing (ATP) therapy, cardiac resynchronization therapy (CRT), bradycardia therapy, or other anti-arrhythmia therapy). As can be seen in FIG. 2, the LCP 100 may be a compact device with all components housed within the LCP 100 or directly on the housing 120. In the example shown in FIG. 2, the LCP 100 may include a communication module 102, a pulse generator module 104, an electrical sensing module 106, a mechanical sensing module 108, a processing module 110, a battery 112, and electrodes 114. The LCP 100 may include more or less modules, depending on the application.

The communication module 102 may be configured to communicate with devices such as sensors, other medical devices, and/or the like, that are located externally to the LCP 100. Communication may use, for example, Bluetooth, Bluetooth Low Energy, Medradio, or other communication protocol, and appropriate circuitry including an antenna may be included in the communication module 102. Communication may instead or in addition make use of any of inductive coupling, optical, acoustic, and/or conducted signals, as are known in the art. Communication may serve many purposes including allowing the LCP to be programmed by an external device, enabling the LCP to report device status and events to an external device, etc.

In the example shown in FIG. 2, the pulse generator module 104 may be electrically connected to a plurality of electrodes 114 and/or 114′. The pulse generator module 104 may be configured to generate electrical stimulation signals, such as by including appropriate circuitry for modulating or multiplying power signals obtained from the battery 112, and outputs may include, for example, pacing pulses or other therapy signals.

In some examples, the LCP 100 may not include a pulse generator module 104, in which case the device 100 may be described as an implantable cardiac monitor. The device 100 may collect data about cardiac electrical activity and/or physiological parameters of the patient and communicate such data and/or determinations to one or more other medical devices via the communication module 102.

In some examples, the LCP 100 may include an electrical sensing module 106, and in some cases, a mechanical sensing module 108. The electrical sensing module 106 may be configured to sense the cardiac electrical activity of the heart. The mechanical sensing module 108 may include one or more sensors, such as an accelerometer, a blood pressure sensor, a heart sound sensor, a blood-oxygen sensor, a temperature sensor, a flow sensor and/or any other suitable sensors that are configured to measure one or more mechanical and/or chemical parameters of the patient. Both the electrical sensing module 106 and the mechanical sensing module 108 may be connected to a processing module 110, which may provide signals representative of the sensed mechanical parameters. Although described with respect to FIG. 2 as separate sensing modules, in some cases, the electrical sensing module 106 and the mechanical sensing module 108 may be combined into a single sensing module, as desired. Other sensors, such as a pulse oximetry module, may be included as desired.

The processing module 110 can be configured to control the operation of the LCP 100. For example, the processing module 110 may be configured to receive electrical signals from the electrical sensing module 106 and/or the mechanical sensing module 108. Based on the received signals, the processing module 110 may determine, for example, occurrences and, in some cases, types of arrhythmias. Based on any determined arrhythmias, the processing module 110 may control the pulse generator module 104 to generate electrical stimulation in accordance with one or more therapies to treat the determined arrhythmia(s). The processing module 110 may further receive information from the communication module 102. In some examples, the processing module 110 may use such received information to help determine whether an arrhythmia is occurring, determine a type of arrhythmia, and/or to take particular action in response to the information. The processing module 110 may additionally control the communication module 102 to send/receive information to/from other devices.

In some examples, the processing module 110 may include a pre-programmed chip, such as a very-large-scale integration (VLSI) chip and/or an application specific integrated circuit (ASIC). In such embodiments, the chip may be pre-programmed with control logic in order to control the operation of the LCP 100. By using a pre-programmed chip, the processing module 110 may use less power than other programmable circuits (e.g., general purpose programmable microprocessors) while still being able to maintain basic functionality, thereby potentially increasing the battery life of the LCP 100. In other examples, the processing module 110 may include a programmable microprocessor. Such a programmable microprocessor may allow a user to modify the control logic of the LCP 100 even after implantation, thereby allowing for greater flexibility of the LCP 100 than when using a pre-programmed ASIC. In some examples, the processing module 110 may further include a memory, and the processing module 110 may store information on and read information from the memory. In other examples, the LCP 100 may include a separate memory (not shown) that is in communication with the processing module 110, such that the processing module 110 may read and write information to and from the separate memory.

The battery 112 may provide power to the LCP 100 for its operations. In some examples, the battery 112 may be a non-rechargeable lithium-based battery. In other examples, a non-rechargeable battery may be made from other suitable materials, as desired. Because the LCP 100 is an implantable device, access to the LCP 100 may be limited after implantation. Accordingly, it is desirable to have sufficient battery capacity to deliver therapy over a period of treatment such as days, weeks, months, years or even decades. In some instances, the battery 112 may be a rechargeable battery, which may help increase the useable lifespan of the LCP 100. In still other examples, the battery 112 may be some other type of power source, as desired.

To implant the LCP 100 inside a patient's body, an operator (e.g., a physician, clinician, etc.), may fix the LCP 100 to the cardiac tissue of the patient's heart. To facilitate fixation, the LCP 100 may include one or more anchors 116. The anchor 116 may include any one of a number of fixation or anchoring mechanisms. For example, the anchor 116 may include one or more pins, staples, threads, screws, helix, tines, and/or the like. In some examples, although not shown, the anchor 116 may include threads on its external surface that may run along at least a partial length of the anchor 116. The threads may provide friction between the cardiac tissue and the anchor to help fix the anchor 116 within the cardiac tissue. In other examples, the anchor 116 may include other structures such as barbs, spikes, or the like to facilitate engagement with the surrounding cardiac tissue.

FIG. 3 depicts an example of another medical device (MD) 200, which may be used alone or in conjunction with an LCP 100 (FIG. 2) in order to detect and/or treat cardiac arrhythmias and other heart conditions. In the example shown, the MD 200 may include a communication module 202, a pulse generator module 204, an electrical sensing module 206, a mechanical sensing module 208, a processing module 210, and a battery 218. Each of these modules may be similar to the modules 102, 104, 106, 108, and 110 of the LCP 100. Additionally, the battery 218 may be similar to the battery 112 of the LCP 100. In some examples, the MD 200 may have a larger volume within the housing 220 than LCP 100. In such examples, the MD 200 may include a larger battery and/or a larger processing module 210 capable of handling more complex operations than the processing module 110 of the LCP 100.

While it is contemplated that the MD 200 may be another leadless device such as shown in FIG. 2, in some instances the MD 200 may include leads such as leads 212. The leads 212 may include electrical wires that conduct electrical signals between the electrodes 214 and one or more modules located within the housing 220. In some cases, the leads 212 may be connected to and extend away from the housing 220 of the MD 200. In some examples, the leads 212 are implanted on, within, or adjacent to a heart of a patient. The leads 212 may contain one or more electrodes 214 positioned at various locations on the leads 212, and in some cases at various distances from the housing 220. Some of the leads 212 may only include a single electrode 214, while other leads 212 may include multiple electrodes 214. Generally, the electrodes 214 are positioned on the leads 212 such that when the leads 212 are implanted within the patient, one or more of the electrodes 214 are positioned to perform a desired function.

In some cases, the one or more of the electrodes 214 may be in contact with the patient's cardiac tissue, such as by implanting the leads 212 by threading through blood vessels of the patient and either into chambers (atria or ventricles) of the heart, or into blood vessels on the heart within which the lead may be anchored. In some cases, the one or more of the electrodes 214 may be positioned substernally or subcutaneously but adjacent the patient's heart, or into an epicardial position on the pericardium or the heart itself. In some cases, the electrodes 214 may conduct intrinsically generated electrical signals to the leads 212, e.g., signals representative of intrinsic cardiac electrical activity. The leads 212 may, in turn, conduct the received electrical signals to one or more of the modules 202, 204, 206, and 208 of the MD 200. In some cases, the MD 200 may generate electrical stimulation signals, and the leads 212 may conduct the generated electrical stimulation signals to the electrodes 214. The electrodes 214 may then conduct the electrical signals and deliver the signals to the patient's heart (either directly or indirectly).

The mechanical sensing module 208, as with the mechanical sensing module 108, may contain or be electrically connected to one or more sensors, such as accelerometers, blood pressure sensors, heart sound sensors, blood-oxygen sensors, acoustic sensors, ultrasonic sensors and/or other sensors which are configured to measure one or more mechanical/chemical parameters of the heart and/or patient. In some examples, one or more of the sensors may be located on the leads 212, but this is not required. In some examples, one or more of the sensors may be located in the housing 220.

While not required, in some examples, the MD 200 may be an implantable medical device. In such examples, the housing 220 of the MD 200 may be implanted in, for example, a thoracic region of the patient. A pectoral or axillary position may be the site of implant, for example. The housing 220 may generally include any of a number of known materials that are safe for implantation in a human body and may, when implanted, hermetically seal the various components of the MD 200 from fluids and tissues of the patient's body.

In some cases, the MD 200 may be an implantable cardiac pacemaker (ICP). In this example, the MD 200 may have one or more leads, for example leads 212, which are implanted on or within the patient's heart. The one or more leads 212 may include one or more electrodes 214 that are in contact with cardiac tissue and/or blood of the patient's heart. The MD 200 may be configured to sense intrinsically generated cardiac electrical signals and determine, for example, one or more cardiac arrhythmias based on analysis of the sensed signals. The MD 200 may be configured to deliver CRT, ATP therapy, bradycardia therapy, and/or other therapy types via the leads 212 implanted within the heart or in concert with the LCP by commanding the LCP to pace. In some examples, the MD 200 may additionally be configured provide defibrillation therapy.

In some instances, the MD 200 may be an implantable cardioverter-defibrillator (ICD). In such examples, the MD 200 may include one or more leads implanted within a patient's heart. The MD 200 may also be configured to sense cardiac electrical signals, determine occurrences of tachyarrhythmias based on the sensed signals, and may be configured to deliver defibrillation therapy in response to determining an occurrence of a tachyarrhythmia.

It is contemplated that one or more LCPs 100 and/or one or more MDs 200 may be used in combination as an example medical device system. The various devices 100, 200 may communicate through various communication pathways including using RF signals, inductive coupling, conductive coupling optical signals, acoustic signals, or any other signals suitable for communication. The system may further include and be in communication with a display. The display may be a personal computer, tablet computer, smart phone, laptop computer, or other display as desired. In some instances, the display may include input means for receiving an input from a user. For example, the display may also include a keyboard, mouse, actuatable (e.g., pushable) buttons, or a touchscreen display. These are just examples.

With reference to FIG. 4, it will be appreciated that a human heart is controlled via electrical signals that pass through the cardiac tissue and that can be detected by implanted devices such as but not limited to the LCP's 100 and/or MD's 200 of FIG. 2 or 3. FIG. 4 is a graphical representation of an illustrative electrocardiogram (ECG) 300 and a mechanical signal 302, illustrated and marked as a heart sound signal, showing a temporal relationship between electrical signals of the heart and mechanical indications 302 of contraction of the heart (e.g., heart sounds). As can be seen in the illustrative ECG 300, a heartbeat includes a P-wave that indicates atrial depolarization associated with an atrial contraction to load the ventricles. A QRS complex, including a Q-wave, an R-wave and an S-wave, represents a ventricular depolarization that is associated with the ventricles contracting to pump blood to the body and lungs. A T-wave shows the repolarization of the ventricles in preparation for a next heartbeat. With heart disease, the timing of these individual events may be anomalous or abnormal, and the shape, amplitude and/or timing of the various waves can be different from that shown. It will be appreciated that the ECG 300 may be detected by implanted devices such as but not limited to the LCP 100 and/or MD 200 of FIG. 2 or 3.

A characteristic or event in the ECG 300 may have a correlated event in the mechanical signal 302. The mechanical response is typically delayed because it takes some time for the heart to respond to the electrical signals. It will be appreciated that heart sounds may be considered as an example of mechanical indications of a heart beating. Other illustrative mechanical indications may include, for example, endocardial acceleration or movement of a heart wall detected by an accelerometer in the LCP, acceleration or movement of a heart wall detected by an accelerometer in an SICD, a pressure, pressure change, or pressure change rate in a chamber of the heart detected by a pressure sensor of the LCP or other implantable device, acoustic signals caused by heart movements detected by an acoustic sensor (e.g., accelerometer, microphone, etc.), twisting of the heart detected by a gyroscope in the LCP or other implantable device, and/or any other suitable indication of a heart chamber beating.

Referring to FIG. 4, in some cases, there may be a first heart sound denoted S1 that is produced by vibrations generated by closure of the mitral and tricuspid valves during a ventricular contraction, a second heart sound denoted S2 that is produced by closure of the aortic and pulmonary valves, a third heart sound denoted S3 that is an early diastolic sound caused by the rapid entry of blood from the right atrium into the right ventricle and from the left atrium into the left ventricle, and a fourth heart sound denoted S4 that is a late diastolic sound corresponding to late ventricular filling during an active atrial contraction. These are mechanical responses that can often be detected using various sensors (e.g., microphone, hydrophone, accelerometer, etc.).

Because the heart sounds are a result of cardiac muscle contracting or relaxing in response to an electrical cardiac signal, it will be appreciated that there is typically a delay between the electrical cardiac signal, indicated by the ECG 300, and the corresponding mechanical indication, indicated in the example shown by the heart sounds trace 302. For example, the P-wave of the ECG 300 is the electrical cardiac signal that triggers an atrial contraction of the heart. The S4 heart sound is the mechanical signal caused by the atrial contraction. In some cases, it may be possible to use this relationship between the P-wave and the S4 heart sound. For example, if one of these signals can be detected, their expected timing relationship can be used as a mechanism to search for the other. For example, if the P-wave can be detected, a window following the P-wave can be defined and searched in order to help find and/or isolate the corresponding S4 heart sound. In some cases, detection of both signals may be an indication of an increased confidence level in a detected atrial contraction. In some cases, detection of either signal may be sufficient to identify an atrial contraction. The identification of an atrial contraction may be used to identify an atrial contraction timing fiducial (e.g., a timing marker of the atrial contraction). The QRS signal and the S1 heart sound may be used in a similar manner to identify a ventricular contraction. The identification of a ventricular contraction may be used to identify a ventricular contraction timing fiducial (e.g., a timing marker of the ventricular contraction).

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The disclosure's scope is, of course, defined in the language in which the appended claims are expressed.

What is claimed is:

1. A pacing system configured to sense cardiac activity and to deliver pacing therapy to a patient's heart, the pacing system comprising:
a first electrode configured to be positioned in a first chamber of the heart and configured to deliver a first pacing therapy to the first chamber of the heart;
a second electrode configured to be positioned adjacent to the left atrial appendage of the heart and configured to deliver a second pacing therapy to the left atrial appendage of the heart;
wherein the processing module of the pacing system is configured to time a delivery of at least part of the first pacing therapy of the first electrode based, at least in part on a first timing fiducial and the processing module of the pacing system is configured to time a delivery of at least part of the second pacing therapy of the second electrode based, at least in part on a determined pacing delay between the first pacing therapy and the second pacing therapy; and
wherein the determined pacing delay is configured to maximize a flow of blood into and/or out of the left atrial appendage.

2. The pacing system of claim 1, wherein the first electrode is coupled to a first lead extending from a pacemaker and the second electrode is coupled to a second lead extending from the pacemaker.

3. The pacing system of claim 1, wherein the first electrode is coupled to a first lead extending from a pacemaker and the second electrode is coupled to a leadless cardiac pacemaker.

4. The pacing system of claim 3, wherein the pacemaker is in communication with the leadless cardiac pacemaker.

5. The pacing system of claim 1, wherein the first timing fiducial comprises a heart sound.

6. The pacing system of claim 1, wherein the first timing fiducial comprises a valve opening.

7. The pacing system of claim 1, wherein the first timing fiducial comprises a pressure drop.

8. The pacing system of claim 1, wherein the determined pacing delay is configured to be customized during implantation of the pacing system.

9. The pacing system of claim 1, wherein the first chamber of the heart is a left atrium.

10. A method of implanting a pacing system, the method comprising:
delivering a first electrode to a first chamber of a heart;
delivering a second electrode to a left atrial appendage of the heart;
positioning an echocardiography system to measure blood flow into and/or out of the left atrial appendage of the heart;
delivering a first pacing therapy to the first chamber of the heart;
delivering a second pacing therapy to the left atrial appendage of the heart, the second pacing therapy delivered a period of time after the first pacing therapy;
while delivering the first pacing therapy and the second pacing therapy measuring a flow of blood into and/or out of the left atrial appendage;
repeatedly delivering the first pacing therapy to the first chamber of the heart, the second pacing therapy to the left atrial appendage of the heart and measuring the flow of blood into and/or out of the left atrial appendage while varying the period of time between the first pacing therapy and the second pacing therapy with each iteration of pacing therapy;
selecting an operational period of time between the first pacing therapy and the second pacing therapy which maximizes a flow of blood into and/or out of the left atrial appendage; and
providing pacing therapy to the heart using the operational period of time between the first pacing therapy and the second pacing therapy.

11. The method of claim 10, wherein repeatedly delivering the first pacing therapy to the first chamber of the heart, the second pacing therapy to the left atrial appendage of the heart and measuring the flow of blood into and/or out of the left atrial appendage while varying the period of time between the first pacing therapy and the second pacing therapy with each iteration of pacing therapy is performed at implantation of the first and second electrodes.

12. The method of claim 10, wherein the operational period of time between the first pacing therapy and the second pacing therapy is stored in a processing module in communication with the first electrode and the second electrode.

13. The method of claim 10, wherein varying the period of time between the first pacing therapy and the second pacing therapy comprises incrementally increasing the period of time.

14. The method of claim 10, wherein a timing of the first pacing therapy is based, at least in part, off a first timing fiducial.

15. The method of claim 10, wherein the operational period of time between the first pacing therapy and the second pacing therapy is based, at least in part, off a first timing fiducial.

16. The method of claim 10, wherein repeatedly delivering the first pacing therapy to the first chamber of the heart, the second pacing therapy to the left atrial appendage of the heart and measuring the flow of blood into and/or out of the left atrial appendage while varying the period of time between the first pacing therapy and the second pacing therapy with each iteration of pacing therapy is performed during a configuration mode.

17. The method of claim 10 wherein providing pacing therapy to the heart using the operational period of time between the first pacing therapy and the second pacing therapy occurs in response to a predetermined detected condition.

18. The method of claim 17, wherein the predetermined detected condition is a detected atrial fibrillation and/or a detected pressure drop.

19. A method of implanting a pacing system, the method comprising:
delivering a first electrode to a first chamber of a heart;
delivering a second electrode to a left atrial appendage of the heart;
advancing an echocardiography system adjacent to the left atrial appendage of the heart;
ablating a region of heart tissue between the first electrode and the second electrode;
delivering a first pacing therapy to the first chamber of the heart;
delivering a second pacing therapy to the left atrial appendage of the heart, the second pacing therapy delivered a period of time after the first pacing therapy;
while delivering the first pacing therapy and the second pacing therapy measuring a flow of blood into and/or out of the left atrial appendage;
repeatedly delivering the first pacing therapy to the first chamber of the heart, the second pacing therapy to the left atrial appendage of the heart and measuring the flow of blood into and/or out of the left atrial appendage while varying the period of time between the first pacing therapy and the second pacing therapy with each iteration of pacing therapy;
selecting an operational period of time between the first pacing therapy and the second pacing therapy which maximizes a flow of blood into and/or out of the left atrial appendage; and
providing pacing therapy to the heart using the operational period of time between the first pacing therapy and the second pacing therapy.

20. The method of claim 19, wherein providing pacing therapy to the heart using the operational period of time between the first pacing therapy and the second pacing therapy occurs with each heartbeat.
